(19) United States
(12) Patent Application Publication
(10) Pub. No.: US 2011/0046451 A1
(43) Pub. Date: Feb. 24, 2011

(54) METHOD AND AUTOMATED SYSTEM FOR ASSISTING IN THE PROGNOSIS OF ALZHEIMER'S DISEASE, AND METHOD FOR TRAINING SUCH A SYSTEM

(76) Inventors: Jean Francois Horn, Saint Maur (FR); Marie-Odile Habert, Paris (FR); Bernard Fertil, La Ciotat (FR)

Correspondence Address: GREER, BURNS & CRAIN, 300 S WACKER DR, 25TH FLOOR, CHICAGO, IL, 60606, US

(21) Appl. No.: 12/990,561

(22) PCT Filed: May 14, 2009
(86) PCT No.: PCT/FR09/50905

§ 371 Date: Nov. 1, 2010

(30) Foreign Application Priority Data
May 15, 2008 (FR) 0853158

(51) Int. Cl. A61B 5/00; G06K 9/00

(52) U.S. Cl. 600/300; 382/128

(57) ABSTRACT

A method and automated system for assisting in the prognosis of the progress and for assisting in the diagnosis of Alzheimer's disease in patients suffering from mild cognitive impairment (MCI). Also provided is a method for training such a system related to identifying discriminant regions of the brain and using these regions to fine tune the assistance method, based on new known cases.

Imaging data (PET or SPECT) is used, representing the cerebral activity in a plurality of spatial zones (voxels). The method then includes a normalization processing of the image data, and analysis of the cerebral activity values read in a selection of voxels forming at least one predetermined discriminant region, defined by its coordinates within a spatial reference system.

The invention relates to a method and an automated system for assisting in the prognosis of the progress and for assisting in the diagnosis of Alzheimer's disease in patients suffering from mild memory disorders or mild cognitive impairment (MCI).

It also relates to a method for training such a system related to identifying discriminant regions of the brain and using these regions to fine tune the assistance method, based on new known cases.

The invention can also be used to assist in the diagnosis or prognosis of other neurological diseases or disorders.

Patients suffering from Alzheimer's disease are known to exhibit a preliminary or prodromal phase characterized by mild cognitive impairment, or MCI.

However, such mild cognitive impairment is common among older patients and can be due to numerous causes. Thus, patients suffering from MCI can remain stable (i.e. exhibit little or no progress in their memory disorders over time) or progress towards dementia, in particular Alzheimer's disease.

Whilst new medicaments seek to slow down the progression of Alzheimer's disease, it is becoming increasingly important to be able to make an early diagnosis of this disease in its prodromal phase.

Recently, research has begun to be focussed on new tools such as neuro-imaging and biological markers of the cerebrospinal fluid, which could improve the specificity of the diagnosis of Alzheimer's disease in the prodromal phase.

The imaging tools include in particular Positron Emission Tomography or PET (“PET scan”) or Single-Photon Emission Computed Tomography (SPECT). These are in vivo examination techniques with radioactive tracers providing a functional 3-dimensional digital image obtained as slices, measuring a physiological characteristic representing brain activity. This physiological characteristic may be different from one method to another: SPECT imaging measures the cerebral blood flow, or perfusion, in the brain (123-IAMP, 99 mTc-HMPAO or 99 mTc-ECD), whereas PET imaging measures glucose metabolism (18F-FDG).

For five years, studies have shown that the presence of hypoperfusion or hypometabolism in certain regions of the brain could indicate a probability of conversion to Alzheimer's disease in the following one to three years.

The publication Encinas et al. (2003), Encinas et al. studied an MCI population using SPECT imaging. Cerebral activity was analyzed for 16 anatomical regions of the brain. Within each anatomical region, a statistical test was carried out in order to determine whether the average activity of each region was significantly different between the group with stable MCI and that with converted MCI. Significant differences were thus shown in each of the regions studied. The regions which appeared to be the most discriminant between the groups were: the left frontal and pre-frontal regions, as well as a left parietal region (sensitivity and specificity greater than 0.75).

Hirao et al. (Hirao, Ohnishi et al. 2005) carried out group analyses using SPM99 software in order to determine the most discriminant anatomical regions within a population made up of 24 stable and 52 converted subjects. A reduction in activity within the left angular gyrus, the lower parietal cortex and the precuneus was thus demonstrated in the case of converted MCI. Logistic regression was then used in order to determine the diagnostic value of the extracted regions, with an accuracy of up to 73% for the imaging. This accuracy was then compared with that of neuropsychological tests, which was comprised between 70 and 78% for the neuropsychological tests.

Borroni et al. (Borroni, Anchisi et al. 2006) used principal component analysis (PCA) in order to obtain orthogonal variables. Variance analysis was then used (applied to the PCA components) retaining only the first two canonical variables in order to carry out the classification. These canonical variables are assumed to offer the best separation between the groups. This method made it possible for them to separate 18 converted MCIs and 9 stable MCIs without any errors. They then combined the imaging and the neuropsychological tests by canonical analysis of the correlations. No improvement was achieved since 5 errors were committed (2 on the stable individuals and 3 on the converted individuals). Moreover, during their analysis of the group using SPM, hypoperfusion was demonstrated within the anatomical regions of the upper and lower parietal cortex, as well as of the precuneus in the case of the converted MCIs.

Huang et al. (Huang, Wahlund et al. 2003) used logistic regression according to a similar approach. They also carried out a group analysis using the SPM99 software, between 54 stable MCIs and 23 converted MCIs. They demonstrated a reduction in activity within the left and right parietal cortex. A logistic regression was then used in order to evaluate the discriminant power of the imaging and neuropsychological tests after anatomical segmentation of each examination into 46 volumes of interest using BRASS software. An area under the ROC curve of 0.75 was thus obtained for the imaging and comprised between 0.75 and 0.77 for the different neuropsychological tests. Finally, the imaging was combined with the neuropsychological tests. The performances were thus improved (area under the ROC curve comprised between 0.82 and 0.84).

Moreover, the publication Jean-Francois Horn et al. (2007-04-01 ISBI) describes the use of SPECT-type three-dimensional imaging data in order to make a diagnosis. However, this method is based on the anatomical regions and seeks to provide a differential diagnosis between patients already suffering from a proven pathology, in order to differentiate between cases of Alzheimer's and cases of frontotemporal dementia in vivo, without resorting to an autopsy. Thus, this method does not make it possible to carry out a prediction beforehand and therefore does not make it possible to take early preventative or curative actions.

Such results would however gain by an improvement in their accuracy. In particular, they are often below 80% in terms of sensitivity and specificity, which is regarded as a minimum for satisfactory reliability, and has been specified as such by the “Reagan Biomarker Working group on: “Molecular and Biochemical Markers of Alzheimer's disease” since 1998.

Moreover, principal component analysis is a method of projection which modifies the representation space, which comprises a loss of information and can be a source of bias or inaccuracy (“Starting from a set n of objects in a space of p descriptors, its purpose is to find a representation in a reduced space of k dimensions (k<<p) which retains “the best summary”, within the meaning of the maximum projected variance.

Furthermore, SPECT imaging is often considered to be less reliable than PET imaging, due to its lower resolution and its greater measurement variability. Moreover, PET imaging is more complex, more expensive and less common in current practice.

Now, certain authors, for example Dubois et al., 2007, considered that the perfusion activity determined by SPECT imaging was not acceptable as a biological marker for Alzheimer's disease, as it was generally below this prerequisite level of 80%.

### OBJECTIVES OF THE INVENTION

A purpose of the invention is to assist a practitioner in reaching a diagnosis and/or prognosis aimed at identifying a prodromal phase of Alzheimer's disease or predicting the progress of a patient suffering from MCI-type disorders.

According to a first aspect, the invention proposes an automated tool capable of presenting a statistical classification of examination data from such an unknown patient, or locating it among examination data obtained from a reference population of subjects or patients of known progress, as described in the description as well as in the figures below. According to this aspect, the invention thus provides an automated method for processing spatial data representing a physiological characteristic within the brain of a patient, for assisting in the prediction of Alzheimer's disease.

Furthermore, the invention seeks to facilitate the reliable use of the information relating to such a patient of unknown progress, in order to facilitate the work of an individual seeking to establish a reliable diagnosis or take a decision based on the real nature of the disease or the disorders affecting the patient in question.

The invention seeks in particular to improve the performances of the statistical classification obtained, and therefore potentially of the final diagnosis and prognosis of progress, in particular in terms of sensitivity and specificity, and if possible exceeding the value of 80%.

The tool according to the invention thus proposes calculating and presenting a numerical probability basis which can be used for evaluation of the results from a studied patient, and/or making it possible intuitively to compare the results of the studied patient with those from a known and validated reference population.

According to a second aspect, the invention also proposes improving the statistical classification obtained, in particular by a method as described below. According to this aspect, the invention also proposes using at least one discriminant region defined according to spatial coordinates set out below.

Furthermore, according to a third aspect, the invention proposes a combination of several types of examination making it possible to improve the statistical classification obtained, as set out below.

The improvements obtained relate in particular to the field of reliability of prediction, ergonomics and relevance of use by the practitioner of his knowledge and his experience. In particular they make it possible for him to put the new case in perspective in relation to known situations, visually and intuitively and in relation to a knowledge base which is updated and/or fine tuned as the studied cases advance and progress.

Another purpose is to propose a method for validating and fine-tuning the prediction and positioning abilities of such a tool, allowing the validation and/or subsequent improvement of a method or a system carrying out this automation. A purpose is also to keep this progress as close as possible to the real data by minimizing bias or alterations which may be caused or amplified by modelling, for example by modeling which is too simplistic or distorting.

According to a fourth aspect, the invention thus proposes to develop the proposed tool over time and with use, for example based on the progress of the known case or on an increase in the reference population. According to this aspect, the invention proposes adding at least one new patient to the reference population in one or more iterations, and for this purpose comprises an imaging data input for this patient, as well as a recalculation of the discriminant index based on the new reference population.

According to a fifth aspect, the invention also proposes a method making it possible to automate the identification of discriminant regions which can be used in imaging in order to produce the examination data and to fine-tune the use of these regions, as described hereafter. It can thus be easier and more ergonomic to prepare imaging data for calculation of the discriminant data, for example when the tool is first put in place, or for example during a readjustment or a recalibration based on the tool using a new protocol, or a new imaging resolution, or even a new imaging technology measuring a close characteristic or close representative character, such as transition from SPECT imaging to PET imaging.

The invention is based on works of scientific and statistical research by the inventors published elsewhere, aimed in particular at automatically differentiating within the MCI population, the individuals who will exhibit no progress within the 3 years, from the individuals who will progress towards Alzheimer's disease, in particular based on the analysis of SPECT-type scintigraphic images (Single-Photon Emission Computed Tomography) and neuropsychological tests.

This research has lead to the production of a computer software and system using these data in order to provide the practitioners with automated assistance in their diagnosis and prognosis. This tool is arranged so as to be able to integrate new reference patients, or integrate data on progress found in association with examination data from previously unknown patients.

It should be noted that the method and the system according to the invention provide results which do not in themselves constitute a diagnosis, but simply a statistical classification of the data from a patient in relation to data originating from other patients. According to the invention, such a “statistical classification” can take different forms, in particular:
a statistical numerical scale, resulting from the mathematics of probability, making it possible to locate a patient on a continuous scale, and/or
a one- or two-dimensional graphical representation making it possible to locate this patient visually or intuitively in relation to the reference patients.

Such a classification can then be used, for example by an experienced medical practitioner, as an additional decision element in order to decide on a diagnosis based on his experience of this tool.

Automated classifications can of course be carried out based on such a statistical classification, for example by deciding on a zone limit or a scale positioned on a graphical representation of this statistical classification. The system can then automatically provide a classification of the studied patient, in a specific delimited diagnosis or prognosis category within such a scale or graphical representation.

The choice of and the decision on the positioning of such a category limit thus constitutes a later stage in the development of the statistical classification and/or its graphical representation.

Within an overall method of diagnosis using the statistical classification provided by the invention, the deductive medical phase comprising the assignment of the results to a clinical presentation then corresponds to putting in place such a category limit and to the choice of its positioning in relation to the reference population.

### SUMMARY OF A PREFERRED EMBODIMENT

More particularly, the invention proposes a preferred embodiment, originating from the inventors' research work, which is described in detail below.

This embodiment comprises a computer system running statistical processing and automatic classification software, making it possible to differentiate more easily and with better reliability, within a population at risk (Mild Cognitive Impairment=MCI), patients having no or little risk of progress, so-called “stable” patients, from patients progressing towards Alzheimer's disease, so-called “converted” patients. For this purpose it is based on information extracted from brain images, here of the SPECT type, and from neuropsychological tests. The method is based on a training technique preceded by pre-processing of the images. This method comprises in particular the following steps:
Acquisition of the SPECT images of the brain;
Spatial readjustment of the images;
Extraction of regions of interest;
Taking into account the left symmetric of the extracted regions;
Image intensity normalization in relation to the overall activity of the cortex;
Calculation of the average activity within the extracted regions for each image;
Each patient is thus characterized by two attributes:
The average cerebral activity within the defined regions
A neuropsychological index (Grobert & Buschke Free Recall test)

Firstly, during the setting up or training phase, the software learns to classify the subjects using a database constituted by MCI patients of known progress, i.e. declared stable or converted by a neuropsychologist based on the follow-up examination of the clinical and neuropsychological data.

Secondly, during the use phase, the software provides assistance making it possible to detect, in the case of new MCI subjects, so-called “unknown” patients or patients “of unknown progress”, those presenting a significant risk of conversion to Alzheimer's disease.

This combination of processing of images with automatic extraction of regions of interest, and use of the results of neuropsychological tests, in the context of automated training methods, can possibly be applied to other neurological pathologies.

Other features and advantages of the invention will become apparent from the detailed description of an embodiment which is in no way limitative, and from the attached drawings in which:

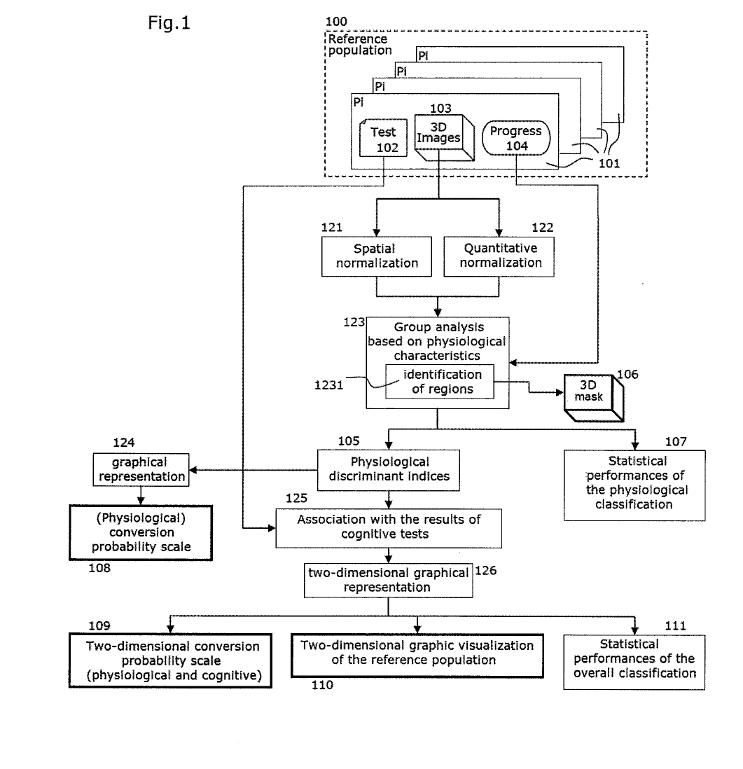

FIG. 1 is a diagram illustrating the set up and the training of software for assistance with the prognosis according to the invention in an embodiment with imaging alone or combined with neuropsychological tests;

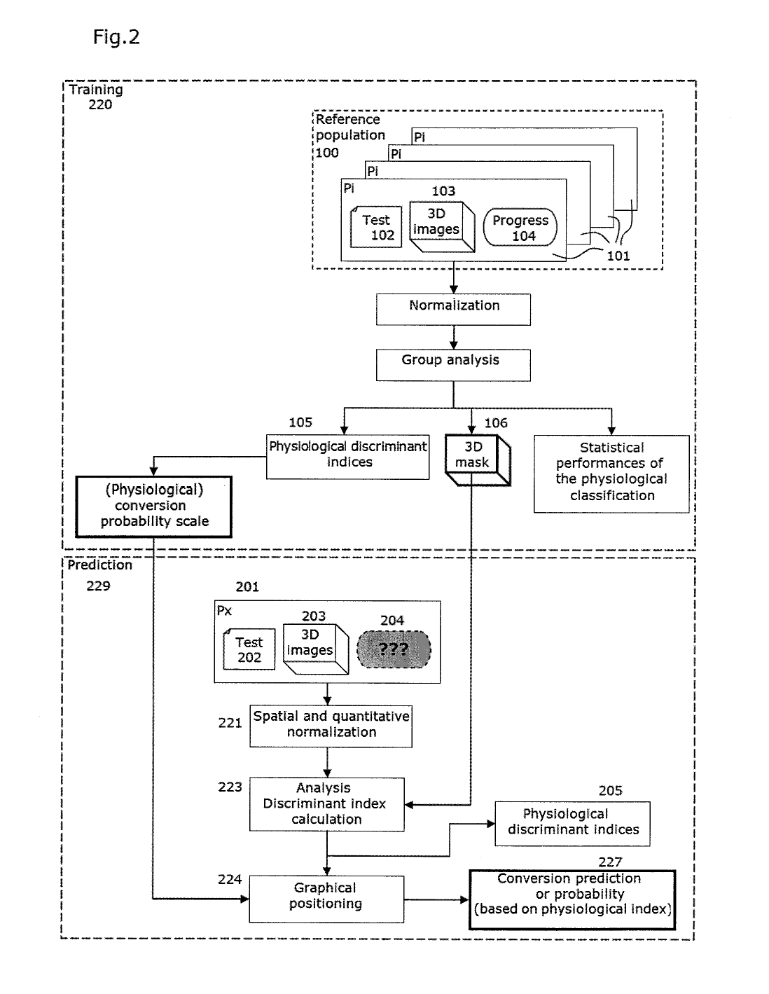

FIG. 2 is a diagram illustrating the set up and the use of software for assisting in prognosis according to the invention in an embodiment with imaging alone;

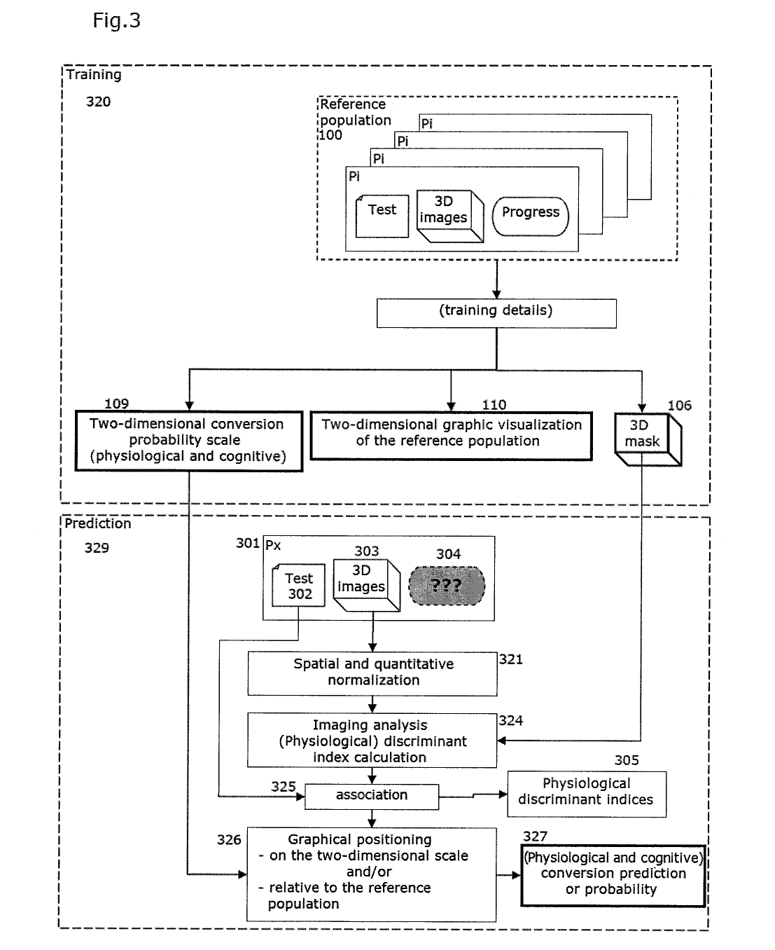

FIG. 3 is a diagram illustrating the set up and the use of software for assisting in prognosis according to the invention in an embodiment with imaging alone and neuropsychological tests;

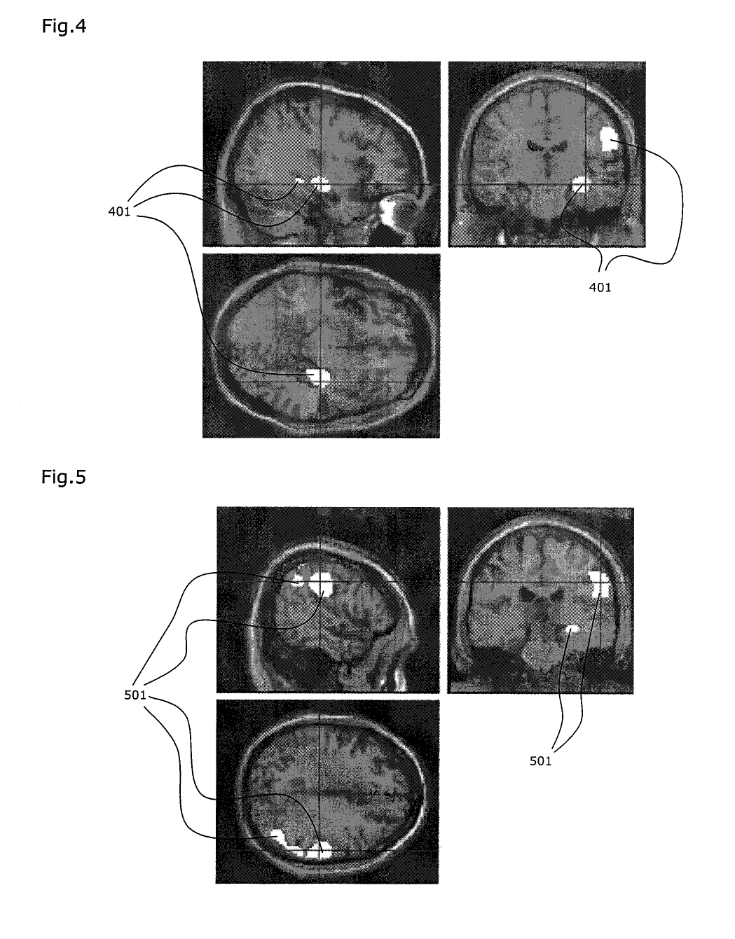

FIG. 4 is a representation in three views of SPECT images, showing a discriminant region close to the right hippocampus, obtained during the set up of software for assisting in prognosis according to the invention;

FIG. 5 is a representation similar to FIG. 4, for an extracted region close to the right parietal cortex;

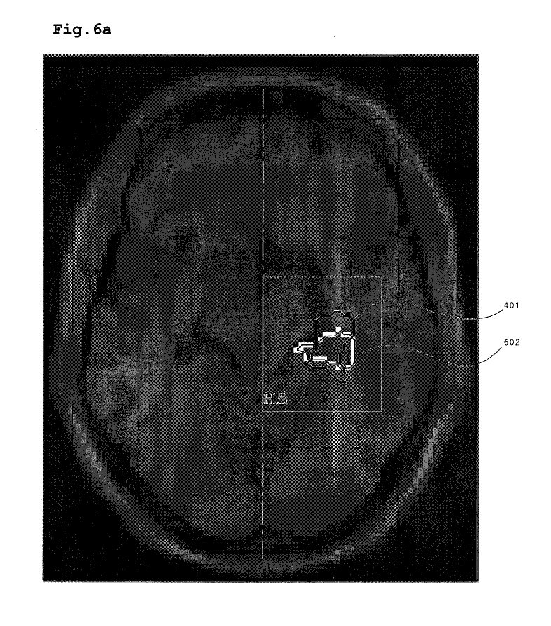

FIG. 6a and FIG. 6b are taken from a tomographical series of SPECT images referenced H1 to H12, in which the following are shown to scale, in a complete view H5 (FIG. 6a) and shown again in detail for all the views (FIG. 6b):
on the one hand, the extracted discriminant region (401) closest to the right hippocampus, obtained during the set up of a software for assisting in prognosis according to the invention,
on the other hand, the anatomical region (602) defined as the right hippocampus;

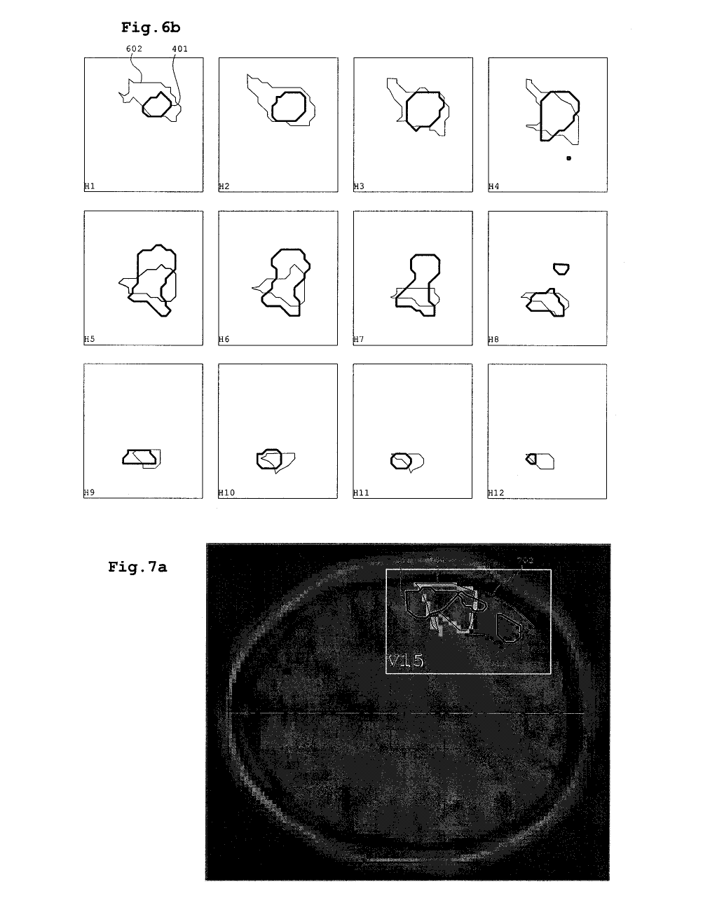

FIG. 7a and FIG. 7b are taken from a tomographical series of SPECT images referenced P1 to P21, in which the following are shown to scale, in a complete view H5 (FIG. 7a) and shown again in detail for all the views (FIG. 7b):
on the one hand, the discriminant region used (501) close to the right parietal cortex,
on the other hand, three close anatomical regions defined as being:
   the “angular gyrus” region (702),
   the “lower parietal cortex” region (703),
   the “supramarginal gyrus” region (704);

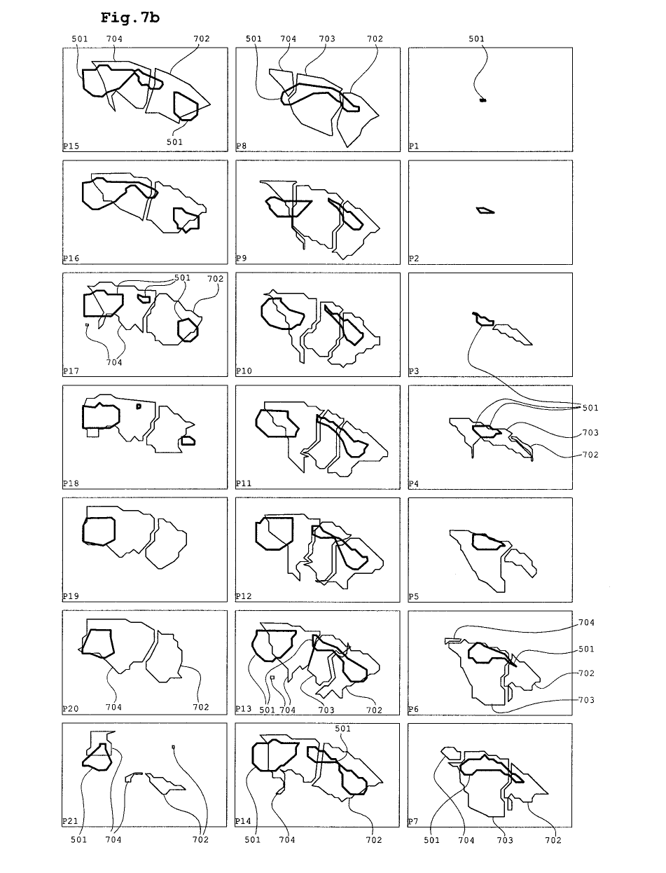

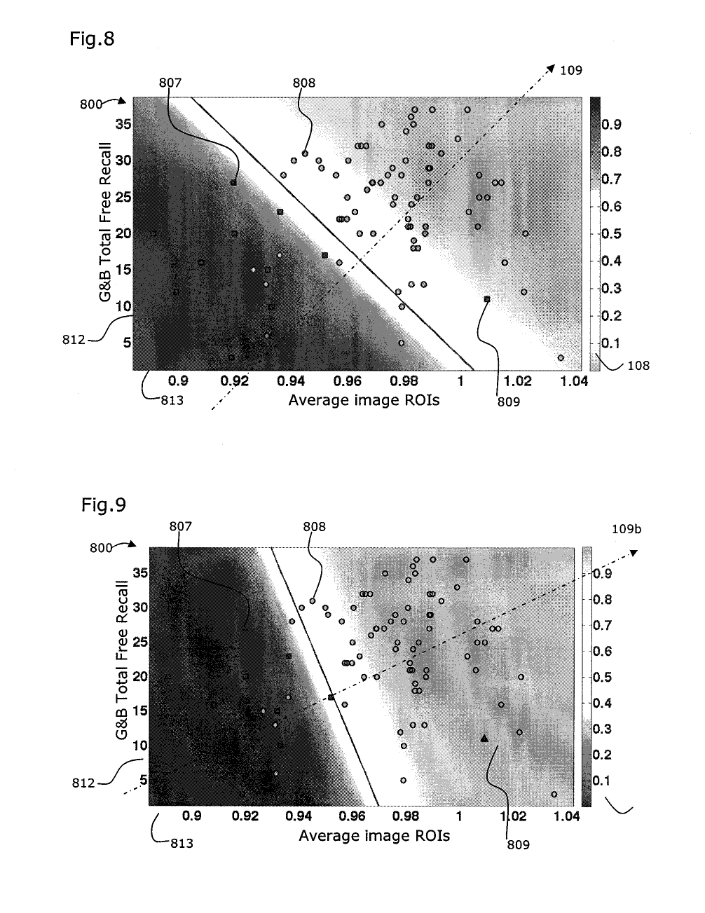

FIG. 8 is a two-dimensional graphical representation of the positioning of a reference population of 83 patients by their SPECT imaging data combined with the “G&B Total Free Recall” test, comprising:
the positioning of the patients,
the scale of colors representing a statistical classification obtained by linear discriminant analysis, and
a decision boundary represented by a separator positioned on the 50% isoprobability line;

FIG. 9 is a graphical representation similar to FIG. 8, for the same population minus one individual (represented by a triangle) considered to be atypical;

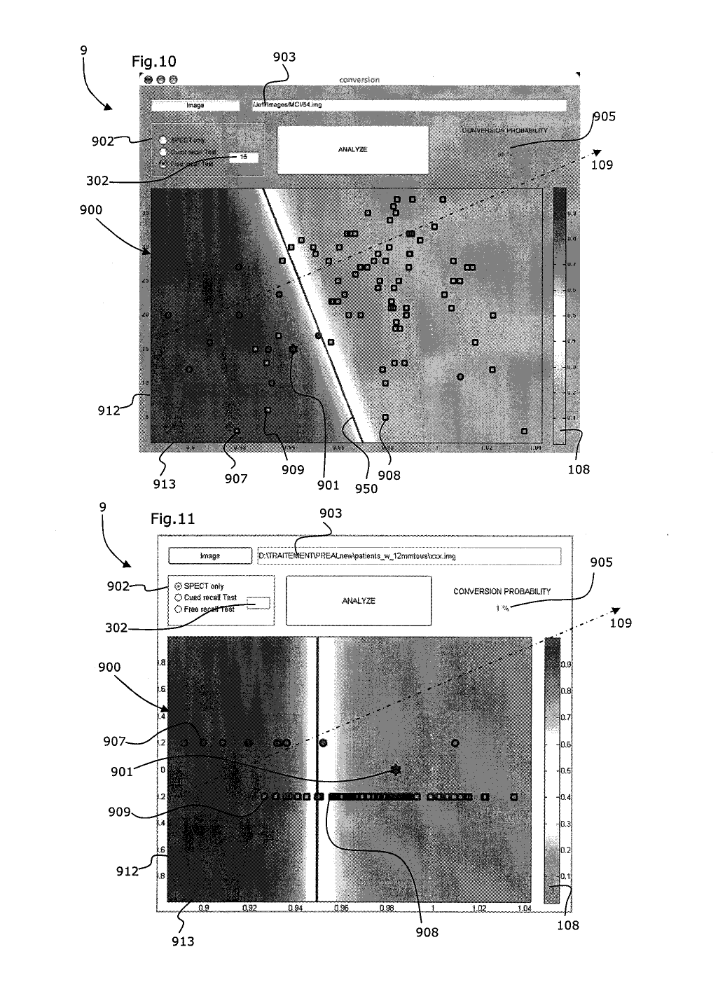

FIG. 10 is a copy of the screen of the interface for assisting with the diagnosis of the software implementing the invention, showing a two-dimensional graphical representation according to FIG. 9 used according to the option of use of SPECT imaging combined with the “G&B Total Free Recall” test;

FIG. 11 is a copy of the screen of the interface for assisting with the diagnosis of the software implementing the invention, showing a two-dimensional graphical representation according to FIG. 9 used according to the option of imaging alone.

### METHODS IMPLEMENTED

The research work on which the present invention is based was carried out with a reference population made up of 83 individuals all diagnosed with MCI at a specific time t₀, and monitored over a period of 3 years. Cerebral scintigraphy following injection of a radioactive tracer, here 99 mTc-ECD, by SPECT imaging was carried out on these patients, as well as a battery of 57 neuropsychological tests. The patients were then monitored over a period of 3 years by a neurologist. We therefore know which patients remained stable at the MCI stage and which have converted to AD.

Part of this work therefore consists of detecting the differences between the two groups of patients, based on the data acquired at time “t₀”, i.e. when included in the study.

When included in the study, the patients all met the clinical criteria for MCI. For 71 of them, this was still the case 3 years later. Twelve of them had progressed towards what is called dementia (also with well-defined clinical criteria), which was Alzheimer's disease in the case of 11 out of the 12.

Reference Population

The reference population suffering from MCI, not corresponding to the clinical diagnostic criteria for dementia, was recruited on the basis of the following criteria:
subjective mnestic disorders detected by means of a questionnaire relating to the experience of the patients vis-à-vis their mnestic disorders in daily activities or concerning recent events;
objective mnestic disorders demonstrated by at least one missing word when recalling three words in the MMSE test (“Mini Mental State Examination”) and/or a score below 29 with the Isaacs set test;
a general retention of the cognitive functions demonstrated by a score above 25 out of 30 in the MMSE test;
a normal score or a single missing object at the first IADL level (“Instrumental Activities of Daily Living”); and
absence of the DSM-IIIR criterion (“Diagnostic and Statistical Manual of Mental Disorders, 3rd edition”) for dementia.

The patients were monitored regularly every 6 months for 3 years. During the monitoring, when a conversion to a dementia was suspected, the diagnosis was again studied by a committee of experts comprising 3 neurologists, 3 neuropsychologists, 3 geriatricians and 3 psychiatrists. They determined whether the clinical criteria for dementia were met using the DSM-IIIR criteria. When a dementia (Alzheimer's) was detected, a complete battery of neuropsychological tests was carried out 6 months later in order to confirm the diagnosis.

A battery of 57 neuropsychological tests was carried out on all the patients when they were included, then annually. These tests included in particular:
free and cued recall tests for verbal episodic memory
the Benton visual retention test for visual memory
the DENO 100 and verbal fluency (letter S and category of fruits in 2 minutes) for language
the serial organization of numbers test and the Baddeley dual-task test for memory functions
the WAIS similarity test for conceptual development
the Stroop test, the Trail Making Test and the WAIS numerical symbols test for the executive functions

Within this population, it proved that only 11 patients out of the 83 present had converted to Alzheimer's disease during the 3 years' monitoring.

Imaging Examination Data

The examination data originating from imaging for different patients, reference or unknown, must be subjected to normalization processes so that they can be compared with each other and utilized. These normalization processes must be similar for the different patients, i.e. they must either be identical or include correctives intended to compensate for the known or found variations, for example depending on the equipment used or the circumstances of data collection.

All or some of the normalization processes described hereafter for the research work are included in the methods according to the invention, as set out in the description of the method for assisting in the prediction and the method for discriminant region determination, and as described below with reference to FIG. 1 to FIG. 3.

Spatial Normalization

Because of variations in the volume and shape of the brain from one individual to another, but also variations in position during acquisition, the images are spatially readjusted so that they are in the same reference system (based on the Talairach reference system) and therefore comparable. The spatial readjustment was carried out using the SPM2 software (Statistical Parametric Mapping) [6, 7]. It consists of applying deformations to the volume so that the anatomical regions of the brain to be readjusted are situated in the same place as those of a reference image, referred to as a “template” (average image produced on the basis of 75 healthy subjects).

First of all, 12 fine-tuned transformations are applied in order to position the original volume in the desired reference system, but also to correct the variations intrinsic to the acquisition. These are 4 types of transformations (translations, rotations, magnifications and stretchings) applied in the 3 dimensions of space. Finally, non-linear deformations are applied to the anatomical regions in order to achieve optimum readjustment.

Quantitative Normalization

After the spatial normalization, and before carrying out the statistical processing, for example the group comparison, each image was smoothed using a Gaussian core (FWHM=12 mm) with the “SPM2” statistical software. Furthermore, before comparing the groups, SPM2 automatically normalizes the images. In order to do this, SPM2 uses the “MRI template” (model used in Magnetic Resonance Imaging) in order to detect the voxels (volumetric pixels) inside the cortex by thresholding the values (in particular with a threshold at 0.8). Then it adapts the scale of the scintigraphic values so that the overall cerebral activity (represented by the perfusion) is 50 ml/min. The age, sex and the imaging centre have also been recorded as variables which may interfere with the analysis.

Determination of the Discriminant Regions

FIG. 1 shows an important characteristic of the invention, which consists of selecting for statistical processing, so-called discriminant cerebral regions which do not necessarily coincide with regions defined anatomically in the state of the art. These “anatomical regions” are defined and named by scientists specializing in the structure of the brain, and have served until now as a minimum unit of analysis for the previous work, for example in the documents of the abovementioned state of the art.

The invention thus proposes using regions defined directly by their spatial position, of a volume which is smaller if possible and corresponding for example to one or more minimum unit volumes (typically the voxels) so that they can be differentiated by the imaging devices used.

These discriminant regions are thus determined and identified as a function of the (known) progress of the patients in the reference population. The definition of these discriminant regions, thus form a three-dimensional “mask” (102, FIG. 1) which is used (123, 223 FIGS. 2 and 323 FIG. 3) by the system according to the invention in order to select, or “extract”, the only imaging data which must be considered to be discriminant: i.e. the imaging data measured in these sole discriminant regions, or “extracted” regions.

According to the invention, an automated method is thus proposed for determination of at least one cerebral region exhibiting a discriminant character for the prediction of conversion to Alzheimer's disease in the patients suffering from mild cognitive impairment (MCI), by means of at least one physiological characteristic of the medium studied measured by imaging in three dimensions, typically perfusion in the case of SPECT images or glucose metabolism in the case of PET images. This discriminant region is identified (1231 FIG. 1) by statistical processing of the imaging data (103) of a reference population (101), and is defined by its spatial coordinates in a specific spatial reference system (typically: Talairach) common to different individuals and providing a system of spatial coordinates within the cerebral volume.

This method of determination comprises the following steps:
for a population 100 of so-called reference patients 101 with the known subsequent progress 104, preparation or acquisition of digital data 103 representing spatial distribution within the brain of at least one quantitative cerebral physiological characteristic (typically: perfusion or metabolism), observed by imaging in three dimensions (typically: SPECT or PET) according to a protocol common to said reference patients;
normalization processing of the imaging data from the different patients, comprising for each of said patients:
   on the one hand, a spatial normalization 121 by readjustment or deformation of the images in order to provide a spatial representation of the brain in accordance with a specific spatial reference system (typically: Talairach) common to the different patients, said reference system providing a system of spatial coordinates within the cerebral volume,
   on the other hand, a quantitative normalization 122 adjusting all of the physiological characteristic values read within the brain of this patient, so as to provide an overall quantitative value of said physiological characteristic in accordance with a specific quantitative reference system, common to the different patients (here: 50 ml/min);
group comparison 123 (here with the SPM2 software) of the values of the physiological characteristic measured between at least two groups of reference patients having experienced different kinds of progress, for each observed spatial zone or voxel, thus providing at least one so-called discriminant region 401, 501 defined by spatial location according to this spatial reference system.

A significance threshold was used based on the t-test values (typically: p<0.05) in order to retain only the most discriminant regions.

FIG. 4 and FIG. 5 represent the two discriminant regions 401 and 501, extracted respectively from the imaging data, in the right hemisphere and in proximity to the anatomical regions of the hippocampus (FIG. 4) and of the parietal cortex (FIG. 5) respectively. These regions are consistent with the topography of the lesions known within Alzheimer's disease.

FIG. 6 and FIG. 7 thus illustrate, for these same two “extracted” discriminant regions 401 and 501 according to the invention, the location of the closest anatomical regions as defined according to the AAL standard (“Anatomical Labeling”) and defined manually on the MRI template of SPM2 (cf. Tzourio-Mazoyer N, Landeau B, Papathanassiou D, Crivello F, Etard O, Delcroix N, et al. anatomical labeling of activations in SPM using a macroscopic anatomical parcellation of the MNI MRI single-subject brain. NeuroImage. 2002; 15:273-89).

For these extracted regions 401 and 501, the close anatomical regions are respectively:
the region 602 of the hippocampus according to its “anatomical” definition, and
the “anatomical” “angular gyrus” region 702, the “lower parietal cortex” region 703 and the “supramarginal gyrus” region 704.

The invention thus proposes using at least one discriminant region having spatial coordinates (according to the atlas proposed by the Montreal Neurological Institute”, close to that of Talairach) including at least one of the following sets of coordinates (the lines in bold represent the most significant part of the region):

| | | | | | | | reg. | |
|---|---|---|---|---|---|---|---|---|
| | | | | | | | according | |
| | | | | MNI | | | to the | |
| Voxel-level | | | | coordinates | | | invention | anatomical regions |
| P_{FDR-corr} | T | Z_{score} | P_{uncorrected} | x | y | z | reference | name & reference |
| 0.041 | 4.21 | 4.01 | 0.000 | 32 | −18 | −12 | 401 | part of the |
| | | | | | | | | hippocampus (602) |
| 0.041 | 3.32 | 3.21 | 0.001 | 32 | −36 | −6 | 401 | outside the |
| | | | | | | | | hippocampus |
| 0.041 | 2.39 | 2.39 | 0.009 | 34 | −4 | −22 | 401 | outside the |
| | | | | | | | | hippocampus |
| 0.041 | 4.04 | 3.87 | 0.000 | 58 | −22 | 36 | 501 | part of the right |
| | | | | | | | | supramarginal gyrus |
| | | | | | | | | (702) |
| 0.041 | 4.01 | 3.84 | 0.001 | 44 | −66 | 36 | 501 | part of the right |
| | | | | | | | | angular gyrus (703) |
| 0.041 | 3.73 | 3.73 | 0.001 | 54 | −46 | 56 | 501 | part of the lower right |
| | | | | | | | | parietal cortex (704) |

P_{uncorrected} indicates the level of significance of the test; P_{FDR-corr} is a corrected value of P_{uncorrected} which takes account of the number of tests carried out; T is the statistical value of the test; x, y and z make it possible to locate the regions in the reference system used.

Thus, the invention proposes using one or more discriminant regions (401, 501) which do not correspond to any of the so-called anatomical regions defined in the state of the art.

More particularly, the invention proposes using at least one discriminant region comprising at least two zones situated in different anatomical regions and comprising less than 75% of each of said different anatomical regions.

In the embodiment described here, such a discriminant region 501 comprises at least three zones situated in the following anatomical regions:
one part less than 75% of the right supramarginal gyrus (702)
one part less than 75% of the right angular gyrus (703), and
one part less than 75% of the lower right parietal cortex (704).

FIG. 6 and FIG. 7 thus illustrate two discriminant regions according to the invention, here called “discriminant region of the hippocampus” 401 and “discriminant region of the right parietal cortex” 501, such that they are identified and used by the invention in the embodiment described here (SPECT imaging). These figures illustrate the location of the anatomical regions closest to the extracted discriminant regions according to the invention. These anatomical regions are respectively the region 602 of the hippocampus according to its “anatomical” definition, and respectively the close “anatomical” regions, i.e. the “angular gyrus” region 702, the “lower parietal cortex” region 703 and the “supramarginal gyrus” region 704.

The regions extracted using SPM2 were defined on our database by a supervised approach taking the desired result into account and may therefore be specific to our data. In order to define the most general methodology possible, we envisaged the use of more general regions, defined independently of our data.

Parallel to the extracted regions, the use of the corresponding anatomical regions was tested and was revealed to be less effective than the regions extracted using SPM2. This comparison is illustrated below relative to the use of the extracted regions.

Set Up and Training of the Computer Tool—Imaging Alone

Using the imaging data from the reference patients, selected for the sole chosen discriminant regions, i.e. according to the mask 106 obtained previously, the patients 101 in the reference population 100 are then characterized on the basis of their examination data 102 and 103, then classified as a function of their subsequent progress 104.

Characterization

The two discriminant regions 401 and 501 in the right hippocampus and in the right parietal cortex were extracted following the group analysis 123 carried out in SPM2, and adopted for the set up of the mask 106.

In the embodiment tested, the regions of the left hemisphere which are symmetrical to the extracted discriminant regions 401 and 501 located in the right hemisphere were included in this mask 106.

Due to the reduced size of the database (83 individuals), an approach by regions, i.e. being able to contain several voxels (independently of the so-called “anatomical” regions), was preferred to an approach by voxels in order to reduce the number of characteristic variables. Thus, the average activity over each of these four regions was calculated. The most effective variables or combinations of variables were determined by means of linear discriminant analysis. We have available the following four variables:
right hippocampus
right parietal cortex
left hippocampus
left parietal cortex

The different combinations of these variables as well as the different combinations obtained by grouping together certain variables (for example, by calculating the average activity over the whole of the left and right hippocampus) can be used in the method according to the invention.

In the preferred embodiment, the adopted combination is a single variable obtained by grouping together, calculating the average activity over the four extracted regions, i.e. the extracted regions in the hippocampus and the parietal cortex, right and left.

In fact, in each region analyzed, the imaging makes it possible to obtain a figure which reflects the average activity in this region, by means of the measured radioactivity. This figure is proportional to the blood flow rate (the perfusion), but does not provide an absolute value for this blood flow rate. It can be expressed by a percentage with respect to the average recorded for the same patient: for example 70% or 120% of the overall average cerebral value of the patient. This figure can also be related to a physiological value, as in the case of the software SPM2 which assigns the standard value of 50 ml/min to the average value (100%) of the activity recorded for each patient, for example in the quantitative normalization phase (122, 221, 321).

By testing each region separately then combined together, it proved that the best results were obtained by taking the average of the average activity of the 4 extracted regions.

Classification

As a function of their subsequent progress 104, the reference patients 101 are classified by a statistical discriminant analysis process, providing a numerical probability scale. The discriminant analysis will calculate the probability that a given individual belongs to each of the classes presented. In order to calculate this probability, the training base information (for example, the average or the variance) was used.

Based on the imaging data 102 from the chosen discriminant regions 401 and 501, this classification provides a first numerical conversion probability scale 108. This scale 108 is referred to as “physiological” and is based on the so-called “physiological” discriminant indices 105 of the reference patients, i.e. obtained only from their imaging data 103. Such a scale can be represented 124 graphically in the form of a one-dimensional scale, for example such as the color-shading scale represented vertically on the right of FIG. 8, FIG. 9 and FIG. 10 and graded from 0 to 1.

The invention thus proposes a computer tool implementing an automated method for developing a scale of probabilities 108 (cf. FIG. 1) of conversion to Alzheimer's disease for patients suffering from mild cognitive impairment (MCI). This method comprises the following steps:
selection or determination of at least one discriminant region 401 and 501 defined by its coordinates in a specific spatial reference system (typically: Talairach), common to different patients 101, 201, 301 and providing a system of spatial coordinates within the cerebral volume;
acquisition or selection of so-called extracted imaging data representing said discriminant region, within numerical data 103 representing a spatial distribution of at least one quantitative cerebral physiological characteristic (typically: perfusion or metabolism), observed by imaging in three dimensions (typically: SPECT or PET) according to a protocol common to said patients;
for a group 100 of the reference patients 101, statistical processing 123 of said extracted data comprising a discriminant analysis providing a numerical function for the calculation of a discriminant index for the conversion prediction, for example a conversion probability, based on the values of the physiological characteristic observed in the discriminant region or regions.

Preferably, this method also comprises, alternatively or successively:
a step of spatial normalization 121 of the imaging data 103, arranged so as to provide for all these patients 101 a spatial representation of the brain according to a specific spatial reference system which is stable from one patient to another, said reference system providing a system of spatial coordinates within the cerebral volume; and/or
a step of quantitative normalization 122 of the imaging data including an adjustment of all of the physiological characteristic values read within the brain of each patient 101, arranged so as to provide an overall quantitative value of said physiological characteristic in accordance with a specific quantitative reference system, common to the different patients (here: 50 ml/min for the perfusion).

The method can then also comprise the calculation of the discriminant indices 105 of a plurality 100 of reference patients 101 from their measured physiological characteristic values, and the graphical positioning 124 of said reference patients on a one-dimensional conversion probability scale 108 based on said discriminant index.

Set Up and Training of the Computer Tool—Combined Imaging and Tests

The inventors' research work also related to a combination of the imaging data with the results of one or more neuropsychological tests.

This combination showed an improvement in the performances achieved and may or may not be included in the tool proposed by the invention.

Two types of test were selected for their performances, from 57 types of neuropsychological tests which were carried out for each patient 101 in the reference population 100. In the same way as for the imaging, a study of the discriminant power of each test was carried out (by monovariable classification) in order to select these two tests.

Preferably, one test is chosen from the two Grober and Buschke tests, i.e. the “free recall” type test or “cued recall” type test.

For each patient 101 individually, the results 102 of this test are associated 125 with the physiological discriminant index 105, in order to be used as coordinates 126 to position this patient on a two-dimensional graphical representation.

As illustrated in FIG. 8, the combination of the results of the tests 103 with the one-dimensional conversion probability scale 108 based on imaging alone then provides a numerical conversion probability scale 109 which can be qualified as “two-dimensional” or “composite”. This means that the numerical values of this probability scale 109 are positioned within a two-dimensional graphical space, here by color variation within a rectangular table 800 according to two perpendicular axes 812 and 813 representing the test data 102 and the discriminant index 105 obtained for the imaging data 103 respectively.

By representing on this two-dimensional scale 109 the associations 125 of data 102 and 103 corresponding to all the reference patients 101, a two-dimensional graphical visualization 110 of the reference population 100 relative to the two-dimensional probability scale 109 is then obtained.

The reference patients 101 are represented in FIG. 8 by the marks 807, 808 positioned in the table 800. The square marks 807 represent those reference patients 101 who have converted to Alzheimer's disease, and the round marks 808 represent those who have remained stable.

In a variant of the invention which may be optional within the computer tool, the discriminant analysis step also relates to a numerical result 103 of at least one and the same neuropsychological, or cognitive, test carried out by each of the reference patients 101.

The invention then also proposes the generation 126 of a graphical representation of a conversion probability scale 109 distributed over two dimensions as a function of, on the one hand, the discriminant index value 105 for the physiological characteristic and, on the other hand, the value of the cognitive test result 103.

Moreover, the invention then proposes the calculation of the discriminant indices of a plurality 100 of reference patients 101 for the imaging data 103 and the graphical positioning 126 of these reference patients on a conversion probability scale 109 distributed over two dimensions as a function of, on the one hand, the discriminant index value 105 for the physiological characteristic and, on the other hand, the value of the cognitive test result 103.

Performances for the Reference Population

During the training of such a computer tool, the invention then also proposes a step of calculation of at least one statistical performance indicator using the “leave-one-out” method. This method of validation consists of successively extracting each individual from the database. The model obtained by training based on all the data except one is then tested on the extracted datum.

An evaluation of the performances of the different options was carried out, comparing for each of the reference patients 101:
on the one hand, their subsequent progress as diagnosed, and
on the other hand, a “virtual” prediction carried out for this patient based on the probability scale 108 or 109 provided by the statistical classification computer tool, considering that a position above the value of 0.5 (50%) was becoming a prognosis towards conversion, and that a position below this corresponded to a prognosis towards stability.

For a single variable obtained by the average of all of the four extracted regions, the following tables show the performances obtained according to the data used.

The following table represents the confusion matrix obtained using linear discriminant analysis for the imaging alone, after leave-one-out validation:

TABLE 1

Virtual prediction

| | | Stable | Converted |
|---|---|---|---|
| Reference | Stable | 63 | 9 |
| | Converted | 1 | 10 |

| | |
|---|---|
| Sensitivity | 0.91 |
| Specificity | 0.88 |
| Accuracy | 0.89 |

Similarly, a classification was carried out considering each of the neuropsychic tests independently of each other. The test achieving the best results is the Grober Buschke (G&B) Free Recall test.

The following table represents the confusion matrix obtained by means of linear discriminant analysis for the G&B Free Recall test, after leave-one-out validation:

TABLE 2

Virtual prediction

| | | Stable | Converted |
|---|---|---|---|
| Reference | Stable | 56 | 16 |
| | Converted | 4 | 7 |

| | |
|---|---|
| Sensitivity | 0.64 |
| Specificity | 0.78 |
| Accuracy | 0.76 |

It is found that the imaging alone makes it possible to differentiate the stable from the converted individuals more effectively than the neuropsychological tests by themselves. However, the errors committed in each of the options are not the same (only 4 errors committed on the stable individuals).

The invention thus proposes a combination of the imaging and the neuropsychological tests, which should therefore make it possible to improve the results of each method. The preferred embodiment of the invention thus combines the most effective variable (average activity in the four extracted regions) for the imaging with the most effective neuropsychological test (G&B Free Recall).

In this combination, the following table represents the confusion matrix obtained using linear discriminant analysis, by combination of the most effective variables for the imaging (average of the activity of the four extracted regions) and the neuropsychological tests (G&B Free Recall), after leave-one-out validation:

TABLE 3

Virtual prediction

| | | Stable | Converted |
|---|---|---|---|
| Reference | Stable | 65 | 7 |
| | Converted | 1 | 10 |

| | |
|---|---|
| Sensitivity | 0.91 |
| Specificity | 0.90 |
| Accuracy | 0.90 |

FIG. 9 illustrates a “two-dimensional” numerical conversion probability scale 109b of the same type as that in FIG. 8, but without taking into account a “converted” individual 809, who is particularly atypical within the converted population (red squares) for the following reasons.

This atypical individual exhibits a high level of activity in the regions examined by imaging, activity which is similar to that of stable individuals and not of the other converted individuals. However, the results obtained in the G&B Free Recall test are in accordance with those of the other converted individuals. Furthermore, his clinical evaluation by a neurologist showed that his progress was compatible with Alzheimer's disease. Moreover, this individual was found to be the only converted subject present in a zone of the numerical scale where most of the stable individuals (green circles) are represented. Furthermore, he is far from the group formed by the converted individuals. His influence on the establishment of the decision rule is then very significant: the classes having been readjusted, the classifier will attempt to compensate for the errors committed in the two classes, and will therefore move his decision boundary in order to attempt to classify this individual correctly. In doing so, he commits numerous errors on the stable individuals, errors which could have been avoided if the classifier had abandoned the idea of classifying this individual correctly.

From a purely statistical point of view and in order to prevent an atypical individual from having too great an effect on the prediction rule, it was therefore decided to remove this individual 807 from the database.

In this combination, and after removal of the atypical individual, the following table represents the confusion matrix obtained using linear discriminant analysis, by combining the most effective variables for the imaging (average activity in the four extracted regions) and the neuropsychologic tests (G&B Free Recall), after leave-one-out validation:

TABLE 4

Prediction

| | | Stable | Converted |
|---|---|---|---|
| Reference | Stable | 67 | 5 |
| | Converted | 1 | 9 |

| | |
|---|---|
| Sensitivity | 0.90 |
| Specificity | 0.93 |
| Efficiency | 0.93 |

Thanks to the invention, the classification of the individuals by virtual prediction has been found to be improved.

Among other things, it is noted that the performances achieved by the invention in terms of sensitivity, specificity and efficacy are above the criteria of 0.80 considered necessary.

In particular, these results are better when the specific extracted discriminant regions as described here are used, than when using the present method but with the cerebral regions determined according to their usual anatomical definition.

By way of comparison, the following table represents the confusion matrix obtained by means of linear discriminant analysis using the average of the anatomical regions and G&B Free Recall, after “leave-one-out” validation:

TABLE 5

Prediction

| | | Stable | Converted |
|---|---|---|---|
| Reference | Stable | 62 | 10 |
| | Converted | 2 | 8 |

| | |
|---|---|
| Sensitivity | 0.86 |
| Specificity | 0.90 |
| Accuracy | 0.85 |

Automated System for Assisting with the Prediction

The statistical classifications carried out on this reference population are used as the engine and database of software run by a computer system, implementing a method for assisting with prediction in the case of patients whose progress is not yet known.

In the preferred embodiment, several options are available in the computer system, implementing several alternatives or variants for this method for assisting in the prediction.

Thus, according to the nature of the examination data available for the patient to be predicted, one or other of the options can be chosen.

The system according to the invention then comprises means arranged in order to classify the examination data of a patient optionally:
either based on imaging data alone
or based on imaging data and a type of cognitive test or a type of test to be chosen from a plurality of possible selections.

Optionally, it would also be possible to combine several cognitive tests without exceeding the scope of the invention.

As illustrated in FIG. 8 and FIG. 10, the combination of the results of the tests 103 with the one-dimensional conversion probability scale 108 based on the imaging alone then provides a two-dimensional numerical conversion probability scale 109.

Prediction—Imaging Alone

FIG. 2 illustrates the method implemented by the option of assisting in the prediction 229 with imaging data alone. Once the computer tool has been set up 220, a similar application 223 of this mathematical processing to the imaging data 203 from a patient 201 of progress 204 to be predicted makes it possible to position this patient on the same one-dimensional numerical scale 108 as the reference population 100.

This positioning 224 can be done numerically, simply by obtaining an index 205 on a purely numerical scale. It can also be done visually, by graphically positioning 224 an indicator for this patient to be predicted on the same graphical scale as the patients 101 in the reference population 100.

The positioning of this patient 201 to be predicted thus rapidly and intuitively provides a practitioner or a user with a basis for deciding 227 on a diagnosis or a prognosis of progress, for example on the basis of his experience or according to a clinical strategy.

Thus, the invention proposes an automated method for processing imaging data 203 representing at least one cerebral physiological characteristic in a patient 201 suffering from mild cognitive impairment, or MCI with a view to assisting in predicting the appearance of Alzheimer's disease, or conversion. This comprises the following steps:
acquisition or preparation of numerical data representing a three-dimensional image 203 and quantitatively measuring at least one physiological characteristic (in particular perfusion in SPECT imaging or metabolism in PET imaging) in a plurality of three-dimensional spatial zones, or voxels, within the brain of said patient;
normalization processing 221 of the image data obtained, said processing comprising:
   on the one hand, a spatial normalization of the images obtained, so as to provide a spatial representation of the brain according to a specific spatial reference system which is stable from one patient to another, said reference system providing a system of spatial coordinates within the cerebral volume; and
   on the other hand, a quantitative normalization adjusting all of the physiological characteristic values read within the brain of said patient, so as to provide an overall quantitative value of said physiological characteristic in accordance with a specific quantitative reference system, common to the different patients (here: 50 ml/min);
use of a classification method with supervised training, preferably linear discriminant analysis 223, of the functional characteristic values read in a selection of one or more voxels forming at least one predetermined discriminant region 401, 501, 106, defined by its coordinates within said spatial reference system, said discriminant analysis providing for said patient a value 205 of said physiological characteristic which can be compared with a plurality of reference values read and calculated for reference patients 101 of known progress.

Prediction—Combined Imaging and Tests

FIG. 3 illustrates the method implemented by the option of assisting in the prediction 329 based on examination data comprising imaging data 303 and the results 302 of a neuropsychological cognitive test for the patient 301 of progress 304 to be predicted. Once the computer tool is constituted 320, a similar application 323 of these mathematical processings to the imaging data 303 from the new patient 301 provides a discriminant index 305.

The patient to be predicted can then be positioned 326 on a two-dimensional graphical representation comprising a two-dimensional numerical conversion probability scale 109, on which the reference population 100 can also be recorded.

FIG. 10 illustrates a screen interface of the software implementing the invention. This screen comprises a computer window 9 displaying the two-dimensional probability scale 109 in a two-dimensional graphical representation field 900, in a manner similar to the graphical representation illustrated in FIG. 8 for the set up and training of the assistance tool.

This screen also comprises a box 902 for choosing the prediction option, making it possible to select or not the use of a “Cued Recall” or “Free Recall” type test. This box also comprises an input field receiving the result 302 of the test chosen for the patient to be predicted 301.

In this same screen there is also an input field 903 for the computer path pointing towards a file containing the imaging data 303 from the patient to be predicted 301.

Once the processing of the examination data 301 and 302 has been carried out, the software displays in the graphical field 900 a point 901 (here in the form of a star) representing the patient to be predicted 301 and positioned according to a vertical axis 912 for the test result 302 and a horizontal axis 913 for the discriminant index 305 originating from the imaging data 303.

Within the field, the two-dimensional probability scale 109 as defined previously for the reference population 100 allows easy evaluation (here by the color shading value at the level of the point 901) of a conversion probability value for this new patient 301. This value is also displayed numerically by the software in a display field 905.

The positioning of this patient 301 to be predicted thus rapidly and intuitively provides a practitioner or a user with a basis for deciding 327 on a prognosis or a diagnosis, for example on the basis of his experience or according to a clinical strategy.

As illustrated in the figure, all or some of the reference patients 101 are also positioned in the graphical representation field 900 as a function of their own results. Unlike FIG. 8, it is to be noted that the converted patients are here represented by a square 907, and the stable patients by a circle 908.

This graphical distribution of the reference population 100 thus allows easy and intuitive visualization of the position 901 of the new patient 301 among the positions 907, 908 of the reference patients. This visualization thus makes it possible not only to rapidly locate the distances in relation to each other, but also to simply evaluate whether the patient to be predicted 301 is situated in a graphical zone of the field 900 where the reference patients 101 are numerous or not, which can give an intuitive idea of the specific reliability of this prediction 901. If the patient is alone in a zone populated by few reference patients 101, or populated by reference patients 909 for whom the virtual predictions have proved to be false (here stable patients 909 in a probability zone above the separating line 950 representing the 50% probability value), the operator can intuitively visualize that the prediction provided might not be as reliable as the overall performances globales of the models represented.

It should be noted that the representation of the scale (here the color shading) or the visualization of the reference population can be carried out concurrently or separately, depending on the options or depending on the embodiments.

As illustrated in FIG. 11, this screen can moreover also serve for the calculation or display of a prediction based on imaging alone, for example according to the choice checked by the user in the test selection box 902. When no test is selected, the predicted 201 and reference 101 patients are displayed linearly. In the figure, the reference patients are moreover distributed over several lines in order to show visibly whether these are patients having converted 907 or not 908, 909.

Alternatively, the subjects can be also displayed on the vertical scale 108 which appears on the right, for example in the form of a vertical cursor for the predicted patient 201 and a darker or lighter grey depending on the concentration of reference patients 101 at each level of this scale.

The graphical field 900 can also be simply modified in order to display a vertical uniformity and show the one-dimensional scale 108 on the horizontal axis 913.

Thus, in this option, the method for assisting in the prediction according to the invention also comprises the following steps:
association 327 of the physiological characteristic value 305 obtained for the evaluated patient 301, with at least one quantitative cognitive performance value 302 originating from at least one neuropsychological test carried out by said patient;
graphical positioning 326 of an evaluation 901 of said patient, by his values obtained for the physiological characteristic and for his cognitive performance, in a two-dimensional evaluation space 900 representing:
   the evaluations 907, 908, 909 of a reference population 100 comprising a plurality of patients 101 associated with their progress 104, and/or
   a numerical conversion probability scale 109 originating from linear discriminant analysis 123 relating to said reference population 100, as a function of, on the one hand, its variations 305 for said physiological characteristic and, on the other hand, its variations 302 in cognitive performance.

Complementary Training Over Time

According to the invention, the set up method can then comprise one or more iterations of the addition of at least one new patient of known progress to the reference population 100, comprising, on the one hand, an input of imaging data for said patient and, on the other hand, a recalculation for the new reference population of the numerical function of obtaining the discriminant index for the conversion based on the measured physiological characteristic value.

This training can for example be carried out over time by the operators of the assistance system:
during a probability calculation for a new patient 301, his examination data 302 and 303 are stored in the system; then
when his progress is known or considered to be known, his result 304 is also entered and this patient is integrated in the system's database. A new statistical classification process (cf. FIG. 1) can be launched in order to integrate this patient into the reference population and fine-tune the engine for assisting in recalculating the probability scales 108 and 109.

Of course, the invention is not limited to the examples which have just been described and numerous adjustments can be made to these examples without exceeding the scope of the invention.

1. An automated method for processing numerical imaging data representing at least one cerebral physiological characteristic measured quantitatively in a plurality of three-dimensional spatial zones, or voxels, within the brain of a patient suffering from mild cognitive impairment, or MCI, with a view to assisting in predicting the appearance of Alzheimer's disease, or conversion, said method comprising the following steps:
normalization processing of the image data according to a standard determined as a function of the image data from a population of reference patients of known progress; and
analysis of the values of said physiological characteristic read in a selection of one or more voxels forming at least one predetermined discriminant region, defined by its coordinates within a specific spatial reference system, said analysis providing for said studied patient a value of said physiological characteristic which can be compared with a plurality of reference values read and calculated for said reference patients.

2. The method according to claim 1, characterized in that it relates to numerical data representing a three-dimensional image, the normalization processing comprising the following steps:
on the one hand, a spatial normalization of the image data obtained for the studied patient, so as to provide a spatial representation of the brain according to the same specific spatial reference system which is stable from one patient to another; and
on the other hand, a quantitative normalization adjusting all of the values of the physiological characteristic read within the brain of said studied patient, so as to provide an overall quantitative value of said physiological characteristic in accordance with a specific quantitative reference system which is stable from one patient to another.

3. The method according to claim 1, characterized in that it uses at least one discriminant region comprising at least two zones situated in different anatomical regions and comprising less than 75% of each of said different anatomical regions.

4. The method according to claim 1, characterized in that it uses at least one discriminant region comprising at least three zones situated in the following anatomical regions:
right supramarginal gyrus
right angular gyrus, and
lower right parietal cortex.

5. The method according to claim 1, characterized in that it uses at least one discriminant region having at least one of the following sets of spatial coordinates:

| | | | | | | | reg. | |
|---|---|---|---|---|---|---|---|---|
| | | | | MNI | | | according to | |
| Voxel-level | | | | coordinates | | | the invention | anatomical regions |
| P_{FDR-corr} | T | Z_{score} | P_{uncorrected} | x | y | z | reference | name & reference |
| 0.041 | 4.21 | 4.01 | 0.000 | 32 | −18 | −12 | 401 | part of the hippocampus |
| | | | | | | | | (602) |
| 0.041 | 3.32 | 3.21 | 0.001 | 32 | −36 | −6 | 401 | outside the hippocampus |
| 0.041 | 2.39 | 2.39 | 0.009 | 34 | −4 | −22 | 401 | outside the hippocampus |
| 0.041 | 4.04 | 3.87 | 0.000 | 58 | −22 | 36 | 501 | part of the right |
| | | | | | | | | supramarginal gyrus |
| | | | | | | | | (702) |
| 0.041 | 4.01 | 3.84 | 0.001 | 44 | −66 | 36 | 501 | part of the right angular |
| | | | | | | | | gyrus (703) |
| 0.041 | 3.73 | 3.73 | 0.001 | 54 | −46 | 56 | 501 | part of the lower right |
| | | | | | | | | parietal cortex (704) |

6. The method according to claim 1, characterized in that it also comprises the following steps:
association of the physiological characteristic value obtained for the evaluated patient, with at least one quantitative cognitive performance value originating from a neuropsychological test carried out by said patient;
graphical positioning of an evaluation of said patient, by his values obtained for the physiological characteristic and for his cognitive performance, in a two-dimensional graphical evaluation space representing:
the evaluations of a reference population comprising a plurality of patients associated with their progress, and/or
a numerical conversion probability scale originating from statistical classification processing relating to said reference population, as a function of, on the one hand, its variations for said physiological characteristic and, on the other hand, its variations in cognitive performance.

7. The method according to claim 1, characterized in that the imaging data are obtained by the SPECT or PET method.

8. The method according to claim 6, characterized in that the cognitive test is a “free recall” or “cued recall” type test.

9. The method according to claim 1, characterized in that the classification step comprises linear discriminant analysis.

10. An automated method for developing a scale of probabilities of conversion to Alzheimer's disease for patients suffering from mild cognitive impairment, or MCI, comprising the following steps:
selection or determination of at least one discriminant region defined by its coordinates in a specific spatial reference system, common to different patients and providing a system of spatial coordinates within the cerebral volume;
acquisition or selection of so-called extracted imaging data representing said discriminant region, within numerical data representing a spatial distribution of at least one quantitative cerebral physiological characteristic, observed by imaging in three dimensions according to a protocol common to said patients;
for a group of reference patients, statistical classification processing with supervised training applied to said extracted data and comprising linear discriminant analysis providing a numerical function for the calculation of a discriminant index for the conversion prediction, for example a conversion probability, based on the values of the physiological characteristic observed in said discriminant region or regions.

11. The method according to claim 10, characterized in that it also comprises a step of spatial normalization of the imaging data, to provide for each patient a spatial representation of the brain according to a specific spatial reference system which is stable from one patient to another, said reference system providing a system of spatial coordinates within the cerebral volume.

12. The method according to claim 10, characterized in that it also comprises a step of quantitative normalization of the imaging data including an adjustment of all of the values of the physiological characteristic read within the brain of each patient, to provide an overall quantitative value of said physiological characteristic in accordance with a specific quantitative reference system, common to the different patients.

13. The method according to claim 10, characterized in that it also comprises the calculation of the discriminant indices of a plurality of reference patients based on their values measured for the physiological characteristic, and the graphical positioning of said reference patients on a one-dimensional conversion probability scale based on said discriminant index.

14. The method according to claim 10, characterized in that the discriminant analysis step also relates to a numerical result from at least one and the same neuropsychological, or cognitive, test carried out by each of the reference patients.

15. The method according to claim 14, characterized in that it also comprises the generation of a graphical representation of a conversion probability scale distributed over two dimensions as a function of, on the one hand, the value of the discriminant index for the physiological characteristic and, on the other hand, the value of the cognitive test result.

16. The method according to claim 16, characterized in that it also comprises the calculation of the discriminant indices of a plurality of reference patients for the imaging data and the graphical positioning of these reference patients on a conversion probability scale distributed over two dimensions as a function of, on the one hand, the value of the discriminant index for the physiological characteristic and, on the other hand, the value of the cognitive test result.

17. The method according to claim 10, characterized in that it also comprises a step of calculation of at least one statistical performance indicator using the “leave-one-out” method.

18. The method according to claim 10, characterized in that it comprises one or more iterations of the addition of at least one patient of known progress to the reference population, comprising, on the one hand, an input of imaging data for said patient and, on the other hand, a recalculation for the new reference population of the numerical function of obtaining the discriminant index for the conversion based on the value of the measured physiological characteristic.

19. An automated method for determination of at least one cerebral region exhibiting, by means of at least one physiological characteristic measured by imaging in three dimensions, a discriminant character for the prediction of conversion to Alzheimer's disease in patients suffering from mild cognitive impairment, or MCI, said discriminant region being defined by its spatial coordinates in a specific spatial reference system common to different individuals and providing a system of spatial coordinates within the cerebral volume,
said method comprising the following steps:
for a so-called reference population of patients of known subsequent progress, preparation or acquisition of numerical imaging data representing a spatial distribution within the brain of at least one quantitative cerebral physiological characteristic, observed by imaging in three dimensions according to a protocol common to said reference patients;
normalization processing of the imaging data from the different patients, comprising for each of said patients:
on the one hand, spatial normalization by readjustment or deformation of the images in order to provide a spatial representation of the brain in accordance with a specific spatial reference system common to the different patients, said reference system providing a system of spatial coordinates within the cerebral volume, and
on the other hand, a quantitative normalization adjusting all of the values of the physiological characteristic read within the brain of said patient, so as to provide an overall quantitative value of said physiological characteristic in accordance with a specific quantitative reference system, common to the different reference patients; and
group comparison of the physiological characteristic values measured between at least two groups of reference patients having experienced different progress, for each spatial zone or voxel observed, thus providing at least one so-called discriminant region defined by spatial location according to said spatial reference system.

20. A computer system comprising means arranged for implementing a method according to claim 1.

21. A computer system comprising means arranged for implementing a method according to claim 1, further including means arranged for optionally classifying the examination data from a patient either from imaging data alone or from imaging data and at least one cognitive test.
